(19) United States
(12) Patent Application Publication
(10) Pub. No.: US 2022/0172804 A1
(43) Pub. Date: Jun. 2, 2022

(54) PREDICTING ANIMAL TESTING MEASUREMENT LEVELS

(71) Applicant: INTERNATIONAL BUSINESS MACHINES CORPORATION, ARMONK, NY (US)

(72) Inventors: Laura-Jayne Gardiner, Wirral (GB); Ritesh Vijay Krishna, Sale (GB); Kirk Edmond Jordan, Andover, MA (US)

(21) Appl. No.: 17/108,259

(22) Filed: Dec. 1, 2020

(51) Int. Cl. G16C 20/70; G16C 20/30; G16B 25/00; G01N 33/15

(52) U.S. Cl. CPC G16C 20/70; G01N 33/15; G16B 25/00; G16C 20/30

(57) ABSTRACT

A method, computer system, and a computer program product for predicting animal testing measurement levels is provided. The present invention may include referencing a human gene expression profiles data obtained under in vitro chemical treatment. The present invention may include referencing an animal measurements data obtained under in vivo chemical treatment. The present invention may include assigning an animal measurement level to a human gene expression profile by matching at least one identified compound. The present invention may include creating a chemical fingerprint of the identified compound. The present invention may include creating a machine learning feature space as a function of a human gene expression feature and a chemical fingerprint feature of the identified compound for predicting an associated animal measurement level assigned to the at least one human gene expression profile. The present invention may include training a model using the machine learning feature space.

### BACKGROUND

The present invention relates generally to the field of computing, and more particularly to machine learning methods of training and predicting animal testing measurement levels.

Animal testing in laboratory is used to measure a reaction to chemical compounds before developing and testing the chemical compound on humans. As an example, defining the toxicity potential of a chemical compound is an important step in drug development. Chemical compounds, at certain doses, and times, may cause some toxic effect in a body. If a compound is deemed too toxic for humans, it is avoided. In the laboratory, toxicity is often measured first on animals (often on rats), and if found safe, then in humans. Animal-based toxicity or other reaction testing is expensive and time consuming, so there is a growing demand for computational methods that can help reduce the use of animals for toxicity and other assessments.

### SUMMARY

Embodiments of the present invention disclose a method, computer system, and a computer program product for predicting animal testing measurement levels. The present invention may include referencing a human gene expression profiles data obtained under in vitro chemical treatment. The present invention may include referencing an animal measurements data obtained under in vivo chemical treatment. The present invention may include assigning an animal measurement level to a human gene expression profile by matching at least one identified compound. The present invention may include creating a chemical fingerprint of the identified compound. The present invention may include creating a machine learning feature space as a function of a human gene expression feature and a chemical fingerprint feature of the identified compound for predicting an associated animal measurement level assigned to the at least one human gene expression profile. The present invention may include training a model using the machine learning feature space.

### BRIEF DESCRIPTION OF THE DRAWINGS

The subject matter regarded as the invention is particularly pointed out and distinctly claimed in the concluding portion of the specification. The invention, both as to organization and method of operation, together with objects, features, and advantages thereof, may best be understood by reference to the following detailed description when read with the accompanying drawings.

Preferred embodiments of the present invention will now be described, by way of example only, with reference to the following drawings in which:

FIG. 1 is an operational flowchart illustrating a process for predicting animal testing measurements levels according to at least one embodiment;

FIG. 2 is an operational flowchart illustrating a process for predicting Blood Urea Nitrogen levels according to at least one embodiment;

FIG. 3A illustrates a graph of Blood Urea Nitrogen concentration using example testing data according to at least one embodiment;

FIG. 3B illustrates a graph showing true versus predicted values for test and training data from Blood Urea Nitrogen level prediction according to at least one embodiment;

FIG. 4 is a schematic block diagram of a computer system according to at least one embodiment;

FIG. 5 is a block diagram of an embodiment of a computer system or server according to at least one embodiment;

FIG. 6 is a block diagram of an illustrative cloud computing environment including the computer system depicted in FIG. 4, in accordance with an embodiment of the present disclosure; and

FIG. 7 is a block diagram of functional layers of the illustrative cloud computing environment of FIG. 6, in accordance with an embodiment of the present disclosure.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numbers may be repeated among the figures to indicate corresponding or analogous features.

### DETAILED DESCRIPTION

A method, system, and computer program product are provided for use in predicting animal testing outcomes of treatments involving chemical compounds with the prediction including a translatability of the effect of a chemical compound between human cell lines and animals.

The described method uses human gene expression profiles, obtained under chemical treatments in vitro (e.g., outside of a living organism), to predict an animal measurement in vivo (e.g., inside of a living organism). The animal measurements may relate to findings such as toxicity and side effects in measurements from blood, urine, or other measurable material from the animal. As the human gene expression profiles are generated in vitro, they are cheaper to generate and are available in public databases at mass scale for a range of chemical compounds, administered at different dosage and time-intervals. Therefore, a large and diverse dataset of chemical intervention on human cells can be constructed. Similarly, there is in vivo experimental data available for animals.

Two datasets: a human gene expression dataset of human gene expression profiles obtained under chemical treatments in vitro and an experimental dataset of animal measurements under chemical treatments in vivo are curated and combined to create and train a machine learning model that can be used to predict the effect of a chemical compound on an animal. The machine learning prediction tool can indicate whether there is value in going for animal testing or not, depending on the predictions obtained from the model, and thus reducing the need for excessive animal testing.

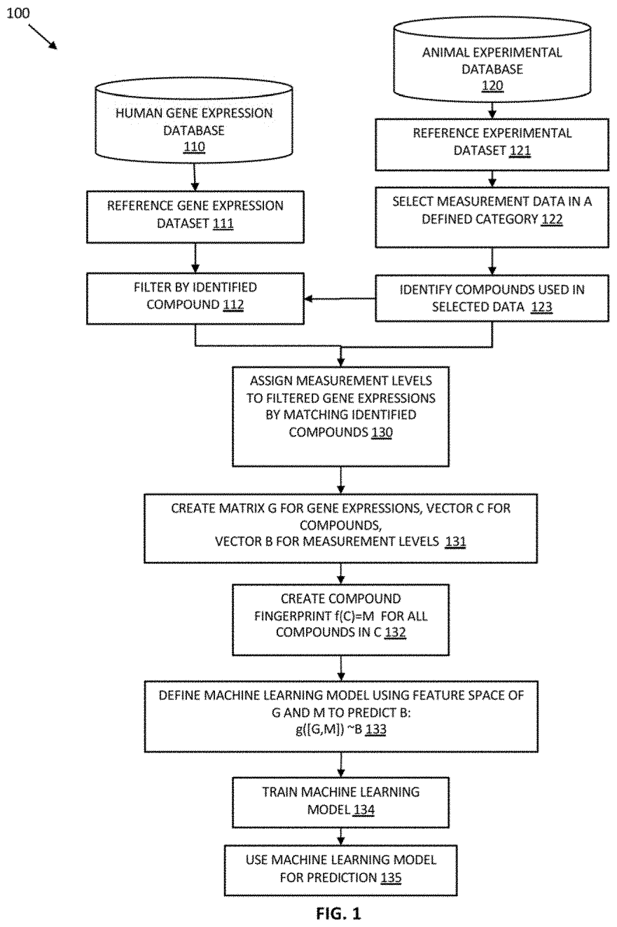

Referring to FIG. 1, an operational flowchart illustrating a process 100 according to at least one embodiment is depicted. Process 100 may be carried out by a computing system to generate and train a machine learning model using a generated feature space and to use the trained machine learning model for prediction of animal testing measurement levels. The process 100 may be provided via a user interface for training a machine learning model and obtaining predictions for a given type of animal experiment.

At 111, process 100 references a human gene expression database 110 of a dataset of human gene expression profiles obtained under chemical treatments in vitro. Humans undergo a cascade of biochemical activities when exposed to a chemical compound. In one embodiment, gene expression measurements may provide a proxy for measuring the underlying biochemical activities. As such, the biochemical dynamics happening underneath may be captured as “digital signals” hidden in gene expression measurements.

According to one embodiment, the human gene expression dataset may be a reduced representation of human transcriptome with measured landmark genes. For example, the dataset may include the human L1000 dataset, where only 964 “landmark genes” need to be measured, and the rest of the human gene expression is computationally extrapolated. This setup has parallels in ‘dimensionality reduction’ techniques, where a reduced representation of data is enough to capture the essence of a larger dataset, and through mathematical extrapolation, one can produce the larger dataset, albeit at the loss of resolution.

At 121, process 100 also references an animal experimental database 120 of a dataset of animal measurements under chemical treatments in vivo. Selected animals that are genetically closely related to humans may provide a good model for testing for drug toxicity, side effects, and other negative or positive reactions. The experimental measurements in animals may represent the underlying biochemical activities caused by the intervention of chemical compounds. For example, blood measurements in rats may be used to show toxicity as certain genetic events happen and Blood Urea Nitrogen (BUN) levels increase.

Then at 122, process 100 selects from the animal experimental dataset, animal measurement data in a defined category, such as measurement levels that meet a defined threshold. The animal experimental dataset may be searched for instances where measurement levels that are reported to be high or low compared to a defined threshold for the measurement. In one example, the animal measurement levels may include blood toxicity levels based on changing blood chemistry under a chemical compound and a defined threshold may include a blood toxicity threshold.

Then at 123, the process 100 identifies compounds used in the chemical treatments that resulted in the selected measurement data.

At 112, process 100 filters from the human gene expression dataset, human gene expression profiles treated by the identified compounds identified from the animal experimental dataset. Compounds may be matched at specific doses and times of administration with animal measurement that were taken at equivalent doses and times. In one embodiment, the compounds must match identically; however, the doses and times may be translated to estimate how dosage and timing in human cells translates to the animal dosage and timing.

Then at 130, process 100 assigns the animal measurement levels from the animal experimental dataset to the filtered human gene expression profiles by matching the identified compounds. The animal measurement levels may be output as a continuous value (e.g., as a numerical scale) to which thresholds may be applied to determine groupings or classifications (e.g., a toxic group). The animal measurement levels may alternatively be grouped based on known thresholds and used for a binary predication (e.g., toxic/non-toxic).

In one embodiment, this may provide a relationship between human gene expression profiles obtained under chemical treatments in vitro using the identified compounds and animal measurement levels obtained under chemical treatments in vivo using the identified compounds. This relationship may be used to build and train a model for prediction including a translatability of the effect of a chemical compound between human cell lines and animals.

According to one embodiment, process 100 may create a machine learning feature space as a function of human gene expression features of the filtered human gene expression profiles and the chemical fingerprint features of the identified compounds for predicting associated animal measurement levels assigned to the human gene expression profiles. The associated animal measurement levels may be predicted as a continuous value that may be compared to defined thresholds.

Then at 131, process 100 creates one or more data structures compatible for machine learning including: a matrix (G) of the filtered human gene expression profiles; a vector (C) of the names of the identified compounds translated; and a vector (B) representing the assigned measurement levels to the human gene expression profiles.

Then at 132, process 100 creates chemical fingerprints (f(C)=M) of the identified compounds. According to one embodiment, process 100 may create for each compound, a chemical fingerprint as a function mapping the compound to a binary representation of captured key information of the compound. In one embodiment, process 100 may use a Molecular Access System (MACCS) representation. Molecular fingerprints may be used in areas of chemoinformatics including diversity analysis and similarity searching.

Then at 133, process 100 defines a machine learning model using the feature space of both the matrix (G) of the filtered human gene expression profiles and the chemical fingerprint (f(C)=M) as a function (g) to predict the vector (B) representing the assigned measurement levels to the human gene expression profiles (g([G,M])˜B).

Then at 134, process 100 trains a machine learning model using the feature space and at 135, the process 100 uses the trained machine learning model for prediction of measurement levels that will indicate whether animal experiments are worthwhile to carry out for a chemical treatment.

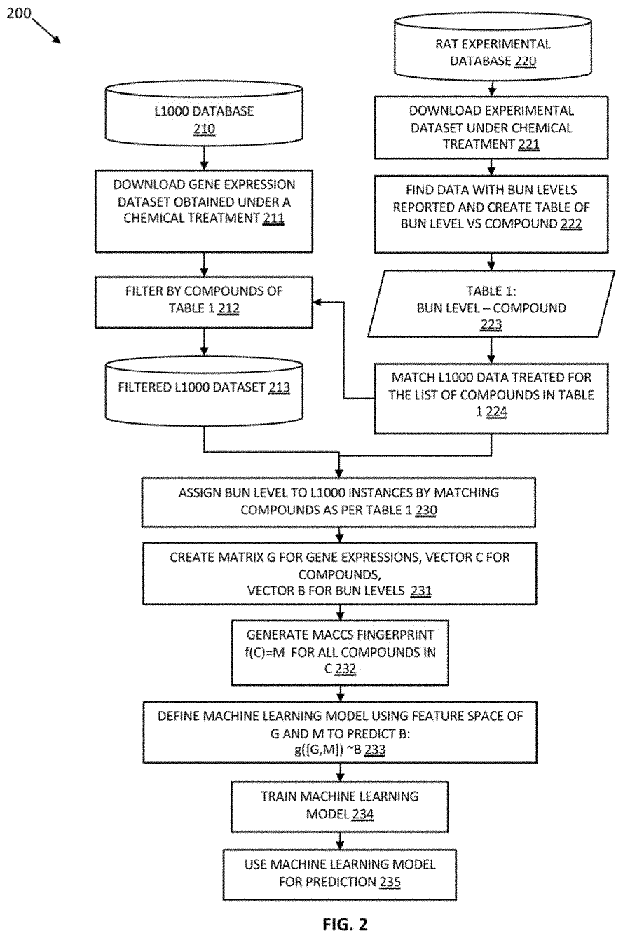

Referring to FIG. 2, an operational flowchart illustrating a process 200 according to at least one embodiment is depicted. Process 200 may be implemented to create a machine learning feature space to predict the Blood Urea Nitrogen (BUN) level in rodents using human (L1000) gene expression datasets for a phenotype, based on toxicity assessment of a chemical compound.

More specifically, process 200 may be implemented to computationally predict, from human L1000 gene expression datasets, a phenotype related to liver and kidney disfunction, known as BUN level in rats and related model species used for toxicity assessment. High BUN levels may be indicative of liver and kidney damage and thus may indicate high toxicity caused by a chemical.

According to one embodiment, L1000 gene expression datasets may be used to computationally predict BUN level in rats and related species for a given compound. L1000 gene expression data along with the computational chemical fingerprint of compounds, may be used for making the prediction. In one embodiment, rats are used as the representative species for toxicity assessment using experimentally obtained blood measurement datasets.

A user interface based software tool may be provided to predict BUN level in rats from human L1000 datasets. A user may interact with the interface to download publicly available human and rat datasets, and instruct the tool to create a machine learning model that may be used for prediction of BUN levels.

At 211, process 200 downloads human L1000 gene expression datasets obtained under a chemical treatment from a publicly available L1000 database 210 and at 221, process 200 downloads rat blood measurement datasets obtained under the chemical treatment from a rat experimental database 220.

Then at 222, process 200 searches the rat datasets to find instances that have BUN levels reported and identifies the compounds used to create pairs of BUN levels and compounds in a Table 1 223.

At 224, the list of compounds obtained in Table 1 (223) is used to match to the human L1000 dataset 211 that were treated by the same list of compounds thereby filtering (at 212) the L1000 dataset 221 for the compounds of Table 1 223 to result in a filtered L1000 dataset 213.

Then at 230, process 200 assigns BUN levels to the filtered human datasets 213, by matching the compound names with BUN levels using Table 1 (223).

Then at 231, process 200 creates data structures compatible for machine learning by creating 1) a matrix G—containing the L1000 gene expression profiles of data in the filtered L100 dataset 213, 2) a vector C—with the names of the chemical compounds, and 3) a vector B—representing the assigned BUN levels.

Then at 232, for each chemical compound in vector C, process 200 generates a corresponding 166 bits MACCS chemical fingerprint, f(C)=M, where f is the function to map compounds to their MACCS representation.

Then at 233, fingerprint and data structures are combined to define a machine learning feature space consisting of both gene expression features and chemical information features as g(G,M)˜B, where g is a function of G and M to predict B.

Then at 234, process 200 trains a machine learning model for the features space to predict the BUN levels and at 235, process 200 uses the trained machine learning model for prediction.

According to one embodiment, rats may be a good model for testing for drug toxicity because they are genetically closely related to humans. In one embodiment, the blood measurements in rats may represent the underlying biochemical activities caused by the intervention of a chemical compound. In case of toxicity, certain genetic events may happen, and BUN levels may increase.

According to one embodiment, humans, being genetically close to rats, may undergo similar (if not the same) cascade of biochemical activities when exposed to a compound. Gene expression measurements may provide a proxy for measuring the underlying biochemical activities. As such, the biochemical dynamics happening underneath may be captured as “digital signals” hidden in gene expression measurements.

The reduced representation human gene expression of the L1000 dataset may capture enough hidden signals to be able to successfully predict BUN levels. This process may be used to predict BUN levels as that may be enough to decide if an animal trial is needed for a compound or not.

For example, the following data sample was used to verify a machine learning model implementing process 200 with the described feature curation for machine learning:

Gene expression features=964;

Chemical fingerprint features=166;

Total features=1130;

Total chemical compounds=429;

Total BUN measurements=429;

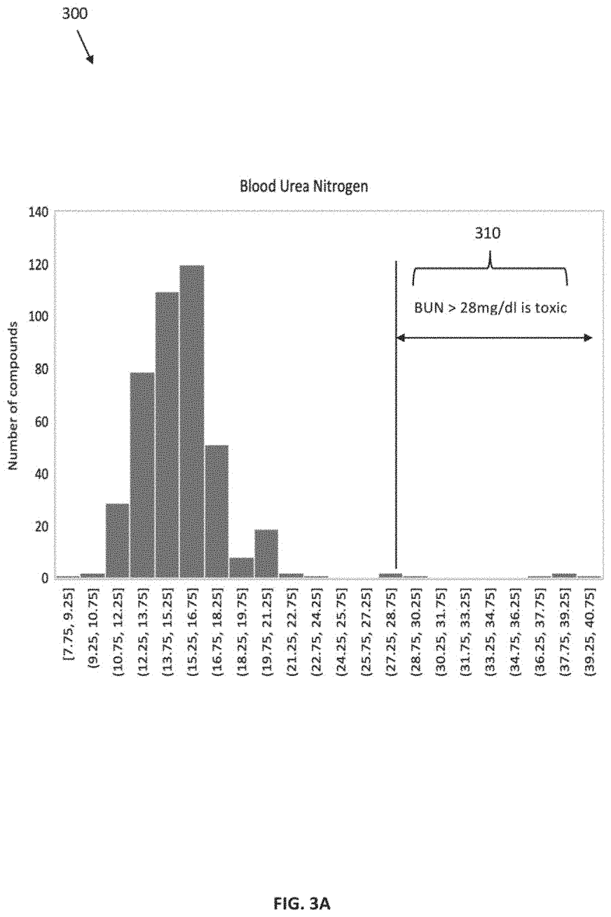

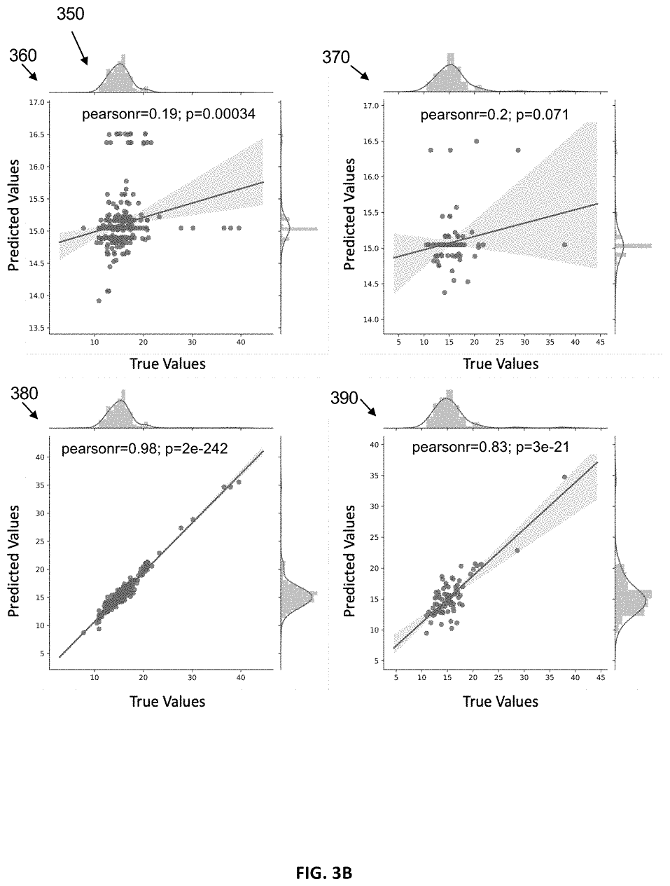

FIG. 3A illustrates a graph 300 of BUN concentration ranges along the x-axis against a number of compounds on the y-axis with a range 310 of BUN >28 mg/dl being toxic.

FIG. 3B illustrates graph results 350 of true versus predicted values for test and training data from BUN level prediction. Scatter plots illustrates the true (x-axis) versus predicted (y-axis) values.

A first pair of graphs illustrate the training dataset 360 and the test dataset 370, predicted using L1000 gene expression data as the only training data and illustrating the best performing parameter set (e.g., using Random Forest Regressor).

A second pair of graphs illustrate the training dataset 380 and the test dataset 390, predicted using L1000 data alongside chemical structure information as training data and showing the best performing parameter set (e.g., using LightGBM Regressor). The second pair of graphs show clearly improved prediction results in the test dataset.

All the scatterplots show marginal histograms with the addition of regression and kernel density fits. The size of the 95% confidence interval for the regression estimate is drawn using translucent bands around the regression line.

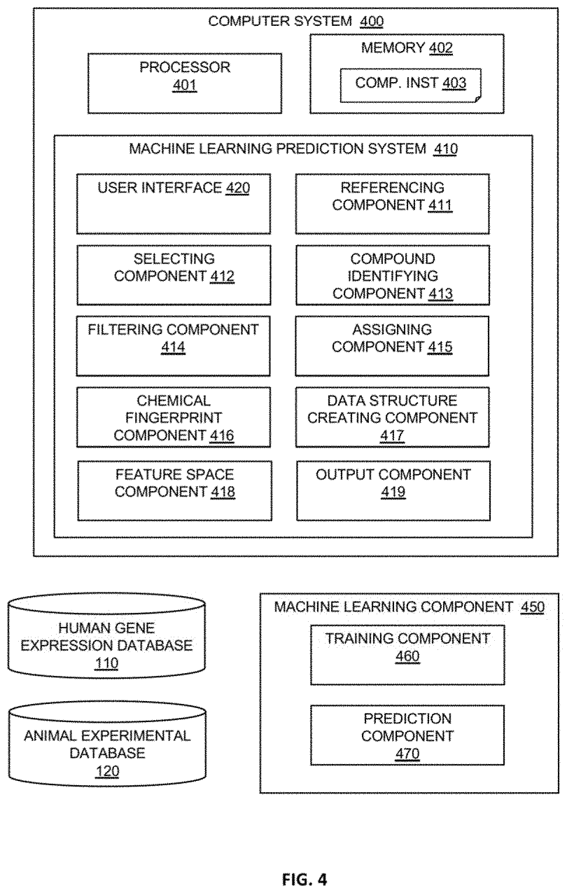

Referring to FIG. 4, a block diagram illustrates an example embodiment of a computer system 400 implementing a machine learning prediction system 410. According to one embodiment, computer system 400 may include a user interface 420 for interacting with the machine learning prediction system 410.

In one embodiment, computer system 400 may include at least one processor 401, a hardware module, or a circuit for executing the functions of the described components which may be software units executing on the at least one processor. Multiple processors 401 running parallel processing threads may be provided enabling parallel processing of some or all of the functions of the components. Memory 402 may be configured to provide computer instructions 403 to the at least one processor 401 to carry out the functionality of the components.

According to one embodiment, the machine learning prediction system 410 may include a referencing component 411 for referencing a human gene expression database 110 of human gene expression profiles obtained under chemical treatments in vitro and referencing an experimental data database 120 of animal measurements under chemical treatments in vivo. In one embodiment, the human gene expression database 110 may be a dataset of reduced representations of human transcriptome with measured landmark genes.

According to one embodiment, the machine learning prediction system 410 may include a selecting component 412 for selecting from the experimental dataset animal measurement data in a defined category, a compound identifying component 413 for identifying compounds used in the chemical treatments of the selected data, and a filtering component 414 for filtering from the human gene expression dataset human gene expression profiles treated by the identified compounds.

According to one embodiment, the machine learning prediction system 410 may include an assigning component 415 for assigning animal measurement levels to human gene expression profiles by matching identified compounds used in the chemical treatments.

According to one embodiment, the machine learning prediction system 410 may include a chemical fingerprint component 416 for creating chemical fingerprints of the identified compounds by creating for each compound a chemical fingerprint as a function mapping the compound to a binary representation of captured key information of the compound.

According to one embodiment, the machine learning prediction system 410 may include a data structure creating component 417 for creating data structures compatible for machine learning and a feature space component 418 for creating a machine learning feature space as a function of human gene expression features of the filtered human gene expression profiles and the chemical fingerprint features of the identified compounds for predicting associated animal measurement levels assigned to the human gene expression profiles.

According to one embodiment, the machine learning prediction system 410 may include an output component 419 for outputting the feature space for training a machine learning model. The output component 419 may interact with a machine learning component 450 including a training component 460 for training a machine learning model. According to one embodiment, the machine learning component 450 may include a prediction component 470 using the trained machine learning model for receiving input data and predicting animal measurement levels assigned to the human gene expression profiles.

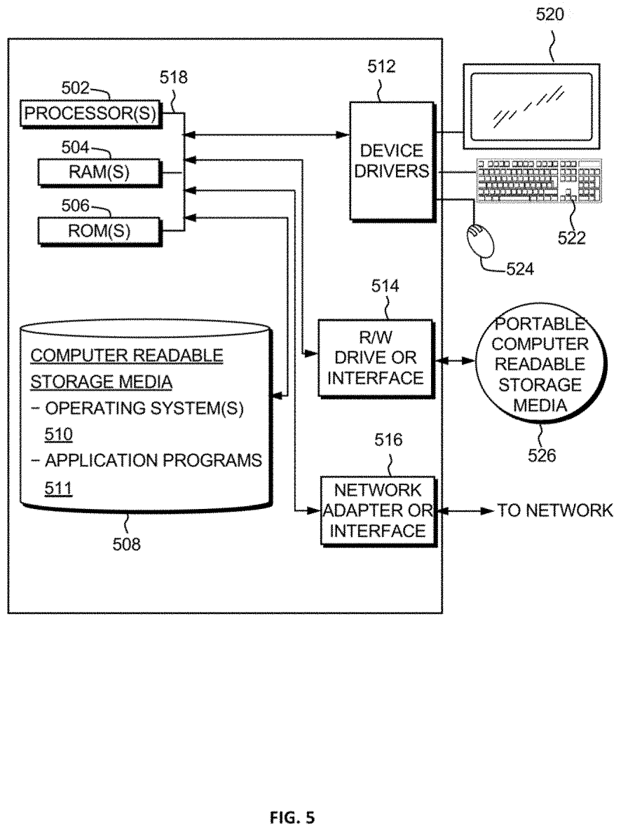

FIG. 5 depicts a block diagram of components of a computing system as used for the machine learning prediction system 410, in accordance with an embodiment of the present invention. It should be appreciated that FIG. 5 provides only an illustration of one implementation and does not imply any limitations with regard to the environments in which different embodiments may be implemented. Many modifications to the depicted environment may be made.

The computing system can include one or more processors 502, one or more computer-readable RAMs 504, one or more computer-readable ROMs 506, one or more computer readable storage media 508, device drivers 512, read/write drive or interface 514, and network adapter or interface 516, all interconnected over a communications fabric 518. Communications fabric 518 can be implemented with any architecture designed for passing data and/or control information between processors (such as microprocessors, communications and network processors, etc.), system memory, peripheral devices, and any other hardware components within the system.

One or more operating systems 510, and application programs 511, are stored on one or more of the computer readable storage media 508 for execution by one or more of the processors 502 via one or more of the respective RAMs 504 (which typically include cache memory). In the illustrated embodiment, each of the computer readable storage media 508 can be a magnetic disk storage device of an internal hard drive, CD-ROM, DVD, memory stick, magnetic tape, magnetic disk, optical disk, a semiconductor storage device such as RAM, ROM, EPROM, flash memory, or any other computer readable storage media that can store a computer program and digital information, in accordance with embodiments of the invention.

The computing system can also include a R/W drive or interface 514 to read from and write to one or more portable computer readable storage media 526. Application programs 511 on the computing system can be stored on one or more of the portable computer readable storage media 526, read via the respective R/W drive or interface 514 and loaded into the respective computer readable storage media 508.

The computing system can also include a network adapter or interface 516, such as a TCP/IP adapter card or wireless communication adapter. Application programs 511 on the computing system can be downloaded to the computing device from an external computer or external storage device via a network (for example, the Internet, a local area network or other wide area networks or wireless networks) and network adapter or interface 516. From the network adapter or interface 516, the programs may be loaded into the computer readable storage media 508. The network may comprise copper wires, optical fibers, wireless transmission, routers, firewalls, switches, gateway computers and edge servers.

The computing system can also include a display screen 520, a keyboard or keypad 522, and a computer mouse or touchpad 524. Device drivers 512 interface to display screen 520 for imaging, to keyboard or keypad 522, to computer mouse or touchpad 524, and/or to display screen 520 for pressure sensing of alphanumeric character entry and user selections. The device drivers 512, R/W drive or interface 514, and network adapter or interface 516 can comprise hardware and software stored in computer readable storage media 508 and/or ROM 506.

The present invention may be a system, a method, and/or a computer program product at any possible technical detail level of integration. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, Python, C++, or the like, and procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be accomplished as one step, executed concurrently, substantially concurrently, in a partially or wholly temporally overlapping manner, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

Cloud Computing

It is to be understood that although this disclosure includes a detailed description on cloud computing, implementation of the teachings recited herein are not limited to a cloud computing environment. Rather, embodiments of the present invention are capable of being implemented in conjunction with any other type of computing environment now known or later developed.

Cloud computing is a model of service delivery for enabling convenient, on-demand network access to a shared pool of configurable computing resources (e.g., networks, network bandwidth, servers, processing, memory, storage, applications, virtual machines, and services) that can be rapidly provisioned and released with minimal management effort or interaction with a provider of the service. This cloud model may include at least five characteristics, at least three service models, and at least four deployment models.

Characteristics are as follows:

On-demand self-service: a cloud consumer can unilaterally provision computing capabilities, such as server time and network storage, as needed automatically without requiring human interaction with the service's provider.

Broad network access: capabilities are available over a network and accessed through standard mechanisms that promote use by heterogeneous thin or thick client platforms (e.g., mobile phones, laptops, and PDAs).

Resource pooling: the provider's computing resources are pooled to serve multiple consumers using a multi-tenant model, with different physical and virtual resources dynamically assigned and reassigned according to demand. There is a sense of location independence in that the consumer generally has no control or knowledge over the exact location of the provided resources but may be able to specify location at a higher level of abstraction (e.g., country, state, or datacenter).

Rapid elasticity: capabilities can be rapidly and elastically provisioned, in some cases automatically, to quickly scale out and rapidly released to quickly scale in. To the consumer, the capabilities available for provisioning often appear to be unlimited and can be purchased in any quantity at any time.

Measured service: cloud systems automatically control and optimize resource use by leveraging a metering capability at some level of abstraction appropriate to the type of service (e.g., storage, processing, bandwidth, and active user accounts). Resource usage can be monitored, controlled, and reported, providing transparency for both the provider and consumer of the utilized service.

Service Models are as follows:

Software as a Service (SaaS): the capability provided to the consumer is to use the provider's applications running on a cloud infrastructure. The applications are accessible from various client devices through a thin client interface such as a web browser (e.g., web-based e-mail). The consumer does not manage or control the underlying cloud infrastructure including network, servers, operating systems, storage, or even individual application capabilities, with the possible exception of limited user-specific application configuration settings.

Platform as a Service (PaaS): the capability provided to the consumer is to deploy onto the cloud infrastructure consumer-created or acquired applications created using programming languages and tools supported by the provider. The consumer does not manage or control the underlying cloud infrastructure including networks, servers, operating systems, or storage, but has control over the deployed applications and possibly application hosting environment configurations.

Infrastructure as a Service (IaaS): the capability provided to the consumer is to provision processing, storage, networks, and other fundamental computing resources where the consumer is able to deploy and run arbitrary software, which can include operating systems and applications. The consumer does not manage or control the underlying cloud infrastructure but has control over operating systems, storage, deployed applications, and possibly limited control of select networking components (e.g., host firewalls).

Deployment Models are as follows:

Private cloud: the cloud infrastructure is operated solely for an organization. It may be managed by the organization or a third party and may exist on-premises or off-premises.

Community cloud: the cloud infrastructure is shared by several organizations and supports a specific community that has shared concerns (e.g., mission, security requirements, policy, and compliance considerations). It may be managed by the organizations or a third party and may exist on-premises or off-premises.

Public cloud: the cloud infrastructure is made available to the general public or a large industry group and is owned by an organization selling cloud services.

Hybrid cloud: the cloud infrastructure is a composition of two or more clouds (private, community, or public) that remain unique entities but are bound together by standardized or proprietary technology that enables data and application portability (e.g., cloud bursting for load-balancing between clouds).

A cloud computing environment is service oriented with a focus on statelessness, low coupling, modularity, and semantic interoperability. At the heart of cloud computing is an infrastructure that includes a network of interconnected nodes.

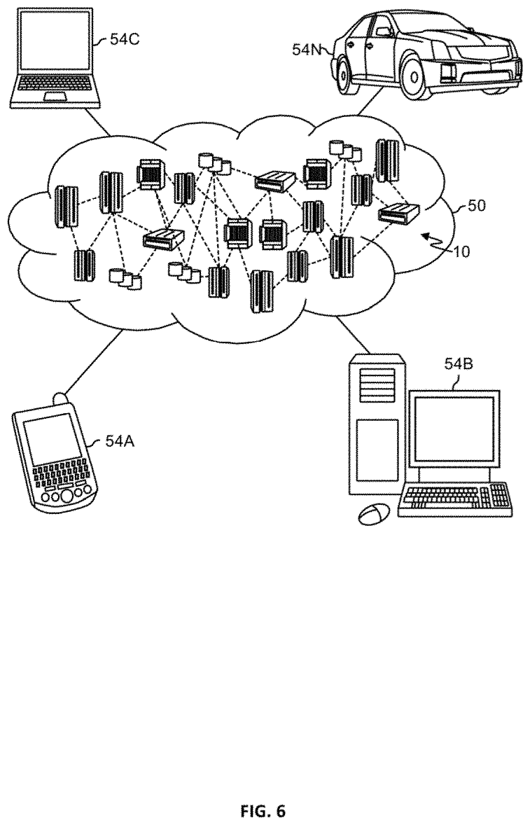

Referring now to FIG. 6, illustrative cloud computing environment 50 is depicted. As shown, cloud computing environment 50 includes one or more cloud computing nodes 10 with which local computing devices used by cloud consumers, such as, for example, personal digital assistant (PDA) or cellular telephone 54A, desktop computer 54B, laptop computer 54C, and/or automobile computer system 54N may communicate. Nodes 10 may communicate with one another. They may be grouped (not shown) physically or virtually, in one or more networks, such as Private, Community, Public, or Hybrid clouds as described hereinabove, or a combination thereof. This allows cloud computing environment 50 to offer infrastructure, platforms and/or software as services for which a cloud consumer does not need to maintain resources on a local computing device. It is understood that the types of computing devices 54A-N shown in FIG. 6 are intended to be illustrative only and that computing nodes 10 and cloud computing environment 50 can communicate with any type of computerized device over any type of network and/or network addressable connection (e.g., using a web browser).

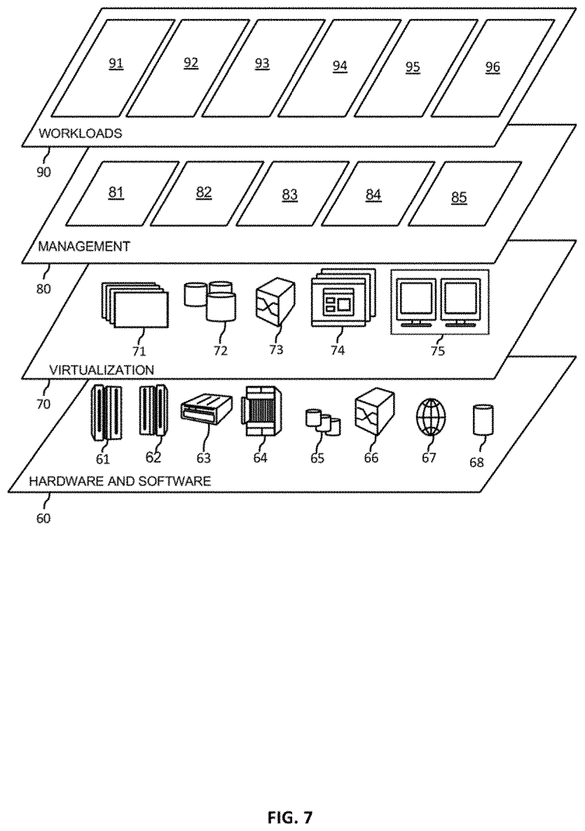

Referring now to FIG. 7, a set of functional abstraction layers provided by cloud computing environment 50 (FIG. 6) is shown. It should be understood in advance that the components, layers, and functions shown in FIG. 7 are intended to be illustrative only and embodiments of the invention are not limited thereto. As depicted, the following layers and corresponding functions are provided:

Hardware and software layer 60 includes hardware and software components. Examples of hardware components include: mainframes 61; RISC (Reduced Instruction Set Computer) architecture based servers 62; servers 63; blade servers 64; storage devices 65; and networks and networking components 66. In some embodiments, software components include network application server software 67 and database software 68.

Virtualization layer 70 provides an abstraction layer from which the following examples of virtual entities may be provided: virtual servers 71; virtual storage 72; virtual networks 73, including virtual private networks; virtual applications and operating systems 74; and virtual clients 75.

In one example, management layer 80 may provide the functions described below. Resource provisioning 81 provides dynamic procurement of computing resources and other resources that are utilized to perform tasks within the cloud computing environment. Metering and Pricing 82 provide cost tracking as resources are utilized within the cloud computing environment, and billing or invoicing for consumption of these resources. In one example, these resources may include application software licenses. Security provides identity verification for cloud consumers and tasks, as well as protection for data and other resources. User portal 83 provides access to the cloud computing environment for consumers and system administrators. Service level management 84 provides cloud computing resource allocation and management such that required service levels are met. Service Level Agreement (SLA) planning and fulfillment 85 provide pre-arrangement for, and procurement of, cloud computing resources for which a future requirement is anticipated in accordance with an SLA.

Workloads layer 90 provides examples of functionality for which the cloud computing environment may be utilized. Examples of workloads and functions which may be provided from this layer include: mapping and navigation 91; software development and lifecycle management 92; virtual classroom education delivery 93; data analytics processing 94; transaction processing 95; and machine learning training and processing 96.

A computer program product of the present invention comprises one or more computer readable hardware storage devices having computer readable program code stored therein, said program code executable by one or more processors to implement the methods of the present invention.

A computer system of the present invention comprises one or more processors, one or more memories, and one or more computer readable hardware storage devices, said one or more hardware storage device containing program code executable by the one or more processors via the one or more memories to implement the methods of the present invention.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

Improvements and modifications can be made to the foregoing without departing from the scope of the present invention.

What is claimed is:

1. A computer-implemented method, comprising:
referencing a first dataset of human gene expression profiles obtained under at least one in vitro chemical treatment;
referencing a second dataset of animal measurements obtained under at least one in vivo chemical treatment;
assigning an animal measurement level to at least one human gene expression profile by matching at least one identified compound used in the at least one in vitro chemical treatment and the at least one in vivo chemical treatment;
creating at least one chemical fingerprint of the at least one identified compound;
creating a machine learning feature space as a function of a human gene expression feature of a filtered human gene expression profile and a chemical fingerprint feature of the at least one identified compound for predicting an associated animal measurement level assigned to the at least one human gene expression profile; and
training a machine learning model using the machine learning feature space.

2. The method of claim 1, further comprising:
selecting, from the second dataset, an animal measurement data in a defined category;
identifying at least one compound used in the at least one in vivo chemical treatment associated with the selected animal measurement data; and
filtering, from the first dataset, the at least one human gene expression profile treated by the at least one identified compound.

3. The method of claim 1, wherein creating the at least one chemical fingerprint of the at least one identified compound creates, for a respective compound, a chemical fingerprint as a function mapping the respective compound to a binary representation of captured key information of the respective compound.

4. The method of claim 1, wherein the first dataset of human gene expression profiles obtained under the at least one in vitro chemical treatment includes a reduced representation of human transcriptome with measured landmark genes.

5. The method of claim 1, wherein selecting, from the second dataset, the animal measurement data in the defined category further comprises:
selecting the animal measurement data with the animal measurement level that breaches a defined threshold.

6. The method of claim 5, wherein the animal measurement level includes a blood toxicity level based on changing blood chemistry under the at least one in vivo chemical treatment and wherein the defined threshold includes a blood toxicity threshold.

7. The method of claim 1, further comprising:
creating data structures compatible for machine learning including:
a matrix (G) of the filtered human gene expression profile;
a vector (C) of identified compound names; and
a vector (B) representing the assigned animal measurement level to the human gene expression profile.

8. The method of claim 7, wherein creating the at least one chemical fingerprint of the at least one identified compound generates, for a respective compound, a chemical fingerprint (f(C)=M) as a function mapping the respective compound to a corresponding Molecular Access System (MACCS) representation.

9. The method of claim 8, wherein creating the machine learning feature space further comprises:
defining a function (g) of the matrix (G) of the filtered human gene expression profile and the chemical fingerprint (f(C)=M) to predict the vector (B) representing the assigned animal measurement level to the human gene expression profile (g([G,M])˜B).

10. The method of claim 1, wherein creating the machine learning feature space further comprises:
predicting a Blood Urea Nitrogen (BUN) level in rodents using a human (L1000) gene expression dataset for a phenotype, based on toxicity assessment of a chemical compound.

11. A computer system for predicting animal testing measurement levels, comprising:
one or more processors, one or more computer-readable memories, one or more computer-readable tangible storage media, and program instructions stored on at least one of the one or more computer-readable tangible storage media for execution by at least one of the one or more processors via at least one of the one or more memories, wherein the computer system is capable of performing a method comprising:
referencing a human gene expression dataset of human gene expression profiles obtained under at least one chemical treatment in vitro;
referencing an experimental dataset of animal measurements obtained under at least one chemical treatment in vivo;
assigning an animal measurement level to at least one human gene expression profile by matching at least one identified compound used in the at least one chemical treatment in vitro and the at least one chemical treatment in vivo;
creating at least one chemical fingerprint of the at least one identified compound;
creating a machine learning feature space as a function of a human gene expression feature of a filtered human gene expression profile and a chemical fingerprint feature of the at least one identified compound for predicting an associated animal measurement level assigned to the at least one human gene expression profile; and
training a machine learning model using the machine learning feature space.

12. The computer system of claim 11, further comprising:
selecting from the experimental dataset, animal measurement data in a defined category;
identifying at least one compound used in the at least one chemical treatment of the selected animal measurement data; and
filtering from the human gene expression dataset, the at least one human gene expression profile treated by the at least one identified compound.

13. The computer system of claim 11, wherein creating the at least one chemical fingerprint of the at least one identified compound creates, for a respective compound, a chemical fingerprint as a function mapping the respective compound to a binary representation of captured key information of the respective compound.

14. The computer system of claim 11, wherein the first dataset of human gene expression profiles obtained under the at least one in vitro chemical treatment includes a reduced representation of human transcriptome with measured landmark genes.

15. The computer system of claim 11, wherein selecting, from the second dataset, the animal measurement data in the defined category further comprises:
selecting the animal measurement data with the animal measurement level that breaches a defined threshold.

16. The computer system of claim 15, wherein the animal measurement level includes a blood toxicity level based on changing blood chemistry under the at least one in vivo chemical treatment and wherein the defined threshold includes a blood toxicity threshold.

17. The computer system of claim 11, further comprising:
creating data structures compatible for machine learning including:
a matrix (G) of the filtered human gene expression profile;
a vector (C) of identified compound names; and
a vector (B) representing the assigned animal measurement level to the human gene expression profile.

18. The computer system of claim 17, wherein creating the at least one chemical fingerprint of the at least one identified compound generates, for a respective compound, a chemical fingerprint (f(C)=M) as a function mapping the respective compound to a corresponding Molecular Access System (MACCS) representation.

19. The system as claimed in claim 18, wherein creating the machine learning feature space further comprises:
defining a function (g) of the matrix (G) of the filtered human gene expression profile and the chemical fingerprint (f(C)=M) to predict the vector (B) representing the assigned animal measurement level to the human gene expression profile (g([G,M])˜B).

20. A computer program product for predicting animal testing measurement levels, comprising:
one or more computer-readable storage media and program instructions collectively stored on the one or more computer-readable storage media, the program instructions executable by a processor to cause the processor to perform a method comprising:
referencing a first dataset of human gene expression profiles obtained under at least one in vitro chemical treatment;
referencing a second dataset of animal measurements obtained under at least one in vivo chemical treatment;
assigning an animal measurement level to at least one human gene expression profile by matching at least one identified compound used in the at least one in vitro chemical treatment and the at least one in vivo chemical treatment;
creating at least one chemical fingerprint of the at least one identified compound;
creating a machine learning feature space as a function of a human gene expression feature of a filtered human gene expression profile and a chemical fingerprint feature of the at least one identified compound for predicting an associated animal measurement level assigned to the at least one human gene expression profile; and
training a machine learning model using the machine learning feature space.
